(19) United States
(12) Patent Application Publication
(10) Pub. No.: US 2024/0233891 A1
(43) Pub. Date: Jul. 11, 2024

(54) CLINICAL SYSTEM INTEGRATION

(71) Applicant: United States Government as Represented by the Department of Veterans Affairs, Washington, DC (US)

(72) Inventors: Devvrat Malhotra, Canton, MI (US); William Frederick Weitzel, III, Ypsilanti, MI (US); Gabriel Solomon, Ann Arbor, MI (US)

(21) Appl. No.: 18/406,868

(22) Filed: Jan. 8, 2024

Related U.S. Application Data
(60) Provisional application No. 63/437,530, filed on Jan. 6, 2023.

(51) Int. Cl. G16H 10/60

(52) U.S. Cl. CPC G16H 10/60

(57) ABSTRACT

Methods, systems, and apparatuses are described for clinical system integration. For example, a computing device may receive a request based on one or more of a plurality of clinical systems. The computing device may retrieve, based on the request, a first plurality of interface components. The first plurality of interface components may be retrieved from each of the plurality of clinical systems in real-time. The computing device may convert the first plurality of interface components to a second plurality of interface components. The second plurality of interface components may correspond to the computing device.

### CROSS-REFERENCE TO RELATED APPLICATIONS

This application claims the benefit of U.S. Provisional Application No. 63/437,530, filed Jan. 6, 2023, which is incorporated herein by reference in its entirety.

### BACKGROUND

The proliferation of information systems has led to medical uses by non-technical personnel. Disparate systems can provide solutions to individual problems, which may require medical personnel to learn nuances of the disparate systems to provide care. These clinical solutions may improve clinical care, and cause additional barriers to the effective administration of care in real time.

### SUMMARY

It is to be understood that both the following general description and the following detailed description are exemplary and explanatory only and are not restrictive. Methods, systems, and apparatuses for clinical system integration are described. For example, a computing device (e.g., a user device or a server) may receive a request based on one or more of a plurality of clinical systems. The computing device may retrieve, based on the request, a first plurality of interface components. The first plurality of interface components may be retrieved from each of the plurality of clinical systems in real-time. The computing device may convert the first plurality of interface components to a second plurality of interface components. The second plurality of interface components may correspond to the computing device. The first plurality of interface components may be converted to the second plurality of interface components based on a template of the computing device. The first plurality of interface components may comprise an indicator that is preserved in the conversion of the first plurality of interface components to the second plurality of interface components. For example, the indicator in the first plurality of interface components may be present in the second plurality of interface components after the conversion.

This summary is not intended to identify critical or essential features of the disclosure, but merely to summarize certain features and variations thereof. Other details and features will be described in the sections that follow.

### BRIEF DESCRIPTION OF THE DRAWINGS

In order to provide understanding techniques described, the figures provide non-limiting examples in accordance with one or more implementations of the present disclosure, in which:

FIG. 1 illustrates an example system in accordance with one or more implementations of the present disclosure.

FIG. 2 illustrates example interface components in accordance with one or more implementations of the present disclosure.

FIG. 3 illustrates example interface components in accordance with one or more implementations of the present disclosure.

FIG. 4 illustrates an example system in accordance with one or more implementations of the present disclosure.

FIG. 5 illustrates an example method in accordance with one or more implementations of the present disclosure.

FIG. 6 illustrates an example method in accordance with one or more implementations of the present disclosure.

FIG. 7 illustrates an example method in accordance with one or more implementations of the present disclosure.

FIG. 8 illustrates an example system in accordance with one or more implementations of the present disclosure.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. When values are expressed as approximations, by use of the antecedent “about,” it will be understood that the particular value forms another configuration. It will be further understood that the endpoints of each of the ranges are significant both in relation to the other endpoint, and independently of the other endpoint.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a computer (e.g., a special purpose computer), or other programmable data processing apparatus to produce a machine, such that the processor-executable instructions which execute on the computer or other programmable data processing apparatus create a device for implementing the functions specified in the flowchart block or blocks.

This detailed description may refer to a given entity performing some action. It should be understood that this language may in some cases mean that a system (e.g., a computer) owned and/or controlled by the given entity is actually performing the action.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

These processor-executable instructions may also be stored in a non-transitory computer-readable memory or a computer-readable medium that may direct a computer or other programmable data processing instrument to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing instrument to cause a series of operational steps to be performed on the computer or other programmable instrument to produce a computer-implemented process such that the processor-executable instructions that execute on the computer or other programmable instrument provide steps for implementing the functions specified in the flowchart block or blocks.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

The method steps recited throughout this disclosure may be combined, omitted, rearranged, or otherwise reorganized with any of the figures presented herein and are not intend to be limited to the four corners of each sheet presented.

The techniques disclosed herein may be implemented on a computing device in a way that improves the efficiency of its operation. As an example, the methods, instructions, and steps disclosed herein may improve the functioning of a computing device.

Cross-communication and integration of disparate clinical computing systems are challenged because clinical data is not made available in real-time and integration may challenge operation workflows and require significant training. Further, generic software solutions that do not integrate clinic-specific requirements and reduce clinician experience. A similar look and feel of the clinical interface can decrease cognitive load on the clinician. The interoperability of the different software solutions reduces the implementation barrier for healthcare systems.

For example, middleware platforms may get access to real-time data (e.g., data available to the clinician as soon as the data is entered), which can be combined with intelligent software applications (e.g., machine learning, neural networks). This application may relate to the contextual design of software applications around existing clinical infrastructure and operational workflow and may include the use of similar graphical user interfaces (GUI) for software applications (i.e. clinician would find software applications to have a similar ‘look and feel,’ similar graphics, and require minimal training for implementation), which may result in a seamless integration of different software applications into one final product. This integration allows for real-time, or near real-time implementation into the existing clinical workflow. The integration may target specific clinical applications, or specific disease categories. Examples of specific use cases include applications for: obesity, heart failure, chronic kidney disease, etc. The clinical management applications can be built on or configured through a common base. The integration of these disparate software systems can provide aid in diagnosis and decision support for management, as well as improve patient access by improving clinical efficiency.

In some respects, the integration may allow creation of software solutions that can be overlaid seamlessly on the existing systems. The overlay may be augmented or add to interfaces provided to the clinician to provide solutions for a specific clinical need, a particular setting, or combination thereof.

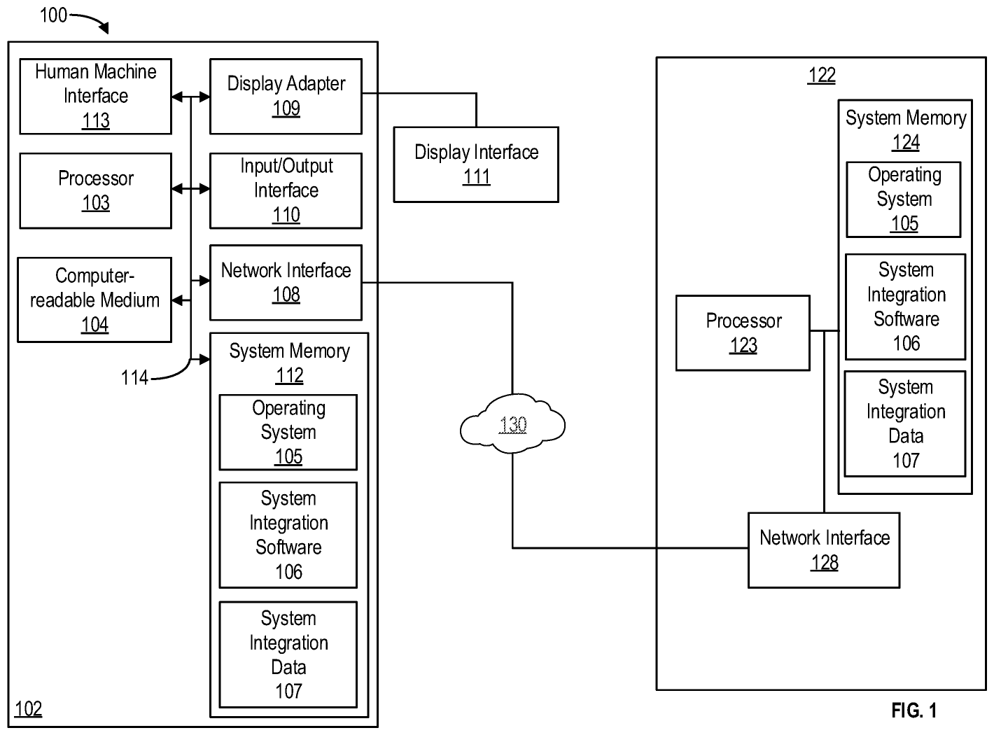

FIG. 1 shows a system 100 in accordance with one or more applications of the present disclosure. The user device 102 may comprise one or more processors 103, a system memory 112, and a bus 114 that couples various components of the user device 102 including the one or more processors 103 to the system memory 112. In the case of multiple processors 103, the user device 102 may utilize parallel computing.

The bus 114 may comprise one or more of several possible types of bus structures, such as a memory bus, memory controller, a peripheral bus, an accelerated graphics port, and a processor or local bus using any of a variety of bus architectures.

The user device 102 may operate on and/or comprise a variety of user device readable media (non-transitory). User device readable media may be any available media that is accessible by the user device 102 and comprises, non-transitory, volatile and/or non-volatile media, removable and non-removable media. The system memory 112 has user device readable media in the form of volatile memory, such as random access memory (RAM), and/or non-volatile memory, such as read only memory (ROM). The system memory 112 may store data such as clinical system integration data 107 and/or programs such as operating system 105 and clinical system integration software 106 that are accessible to and/or are operated on by the one or more processors 103.

The clinical system integration data 107 may comprise a plurality of interface components that correspond to a plurality of clinical systems such as the user device 102. The plurality of interface components may be received from the plurality of clinical systems including the user device 102 via the network interface 108. The plurality of interface components in the clinical system integration data 107 may comprise content such as text, images, and video. The plurality of interface components may comprise borders, framing, infographics, summaries, analytics, indicia, personally identifiable information, and/or the like. The plurality of interface components may comprise patient data. The patient data may include structured data and/or unstructured data. The structured data may include questions to be answered by clinicians, users, or physicians. For example, radio buttons and/or true/false questions may be the form of structured data. The unstructured data may allow the clinicians or physicians to enter patient information in free-form. The unstructured data may comprise one or more of a radiograph, ECG, ultrasound, and/or textual data. The unstructured data may comprise one or more of patient voice notes, clinical staff notes, and textual data.

The clinical system integration software 106 may be implemented by the one or more processor 103. For example, the one or more processor 103 may determine to send a message to the computing device 122 requesting one or more interface components for the user device 102. The request message may be sent to the computing device 122 via the network interface 108. In response to the request, the user device 102 may receive the one or more interface components corresponding to one or more clinical systems. The one or more interface components may be converted from a plurality of interface components that correspond to a plurality of clinical systems. The user device 102 may display patient data (or medical record) specific to the user device 102 (or a specific clinical system) based on the one or more interface components received from the computing device 122.

The user device 102 may also comprise other removable/non-removable, volatile/non-volatile user device storage media. The computer-readable medium 104, e.g., a computer store, may provide non-volatile storage of user device code, user device readable instructions, data structures, programs, and other data for the user device 102. The computer-readable medium 104 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of programs may be stored on the computer-readable medium 104. An operating system 105 and software 106 may be stored on the computer-readable medium 104. One or more of the operating system 105 and software 106 (e.g., mobile applications), or some combination thereof, may comprise program and the software 106. Data 107 may also be stored on the computer-readable medium 104. Data 107 may be stored in any of one or more databases known in the art. The databases may be centralized or distributed across multiple locations within the network 130.

A user may enter commands and information into the user device 102 via an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, touchscreens, and the like. These and other input devices may be connected to the one or more processors 103 via a human machine interface 113 that is coupled to the bus 114, but may be connected by other interface and bus structures, such as a parallel port, game port, an IEEE 1394 Port (also known as a Firewire port), a serial port, network interface 108, and/or a universal serial bus (USB).

A display interface 111 may also be connected to the bus 114 via an interface, such as a display adapter 109. It is contemplated that the user device 102 may have more than one display adapter 109 and the user device 102 may have more than one display interface 111. A display interface 111 may be a monitor, an LCD (Liquid Crystal Display), light emitting diode (LED) display, television, smart lens, smart glass, and/or a projector. In addition to the display interface 111, other output peripheral devices may comprise components such as speakers (not shown) and a printer (not shown) which may be connected to the user device 102 via Input/Output Interface 110. Any step and/or result of the methods may be output (or caused to be output) in any form to an output device. Such output may be any form of visual representation, including, but not limited to, textual, graphical, animation, audio, tactile, and the like. The display interface 111 and user device 102 may be part of one device, or separate devices. The display interface 111 may further allow user input or interaction.

The user device 102 may operate in a networked environment using logical connections to one or more computing devices 122. A computing device 122 may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the user device 102 and a computing device 122 may be made via a network 130. Such network connections may be through a network interface 108. A network interface 108 may be implemented in both wired and wireless environments. The computing device 122 may include one or more processors 123, system memory 124 (e.g., a computer store), and a network interface 128.

For example, the one or more processors 123 may receive a request related to a clinical system (e.g., a clinical system operated by the user device 102) via the network interface 128. For example, the request may be a request for one or more interface components for the user device 102. The request may be sent from the user device 102. Based on the request, the one or more processors 123 may retrieve a first plurality of interface components from the system memory 124 (e.g., system integration data 107). The system integration data 107 of the computing device 122 may comprise a plurality of interface components that correspond to a plurality of clinical systems. The first plurality of interface components may comprise content such as text, images, and video. The first plurality of interface components may comprise borders, framing, infographics, summaries, analytics, indicia, personally identifiable information, and/or the like. The first plurality of interface components may comprise patient data.

Based on the request, the one or more processors 123 may convert the first plurality of interface components to a second plurality of interface components. The first plurality of interface components may be converted to the second plurality of interface components based on a template associated with the computing device 102. The template may indicate sizing, shaping, shading, and coloration of the second plurality of interface components. The template may further comprise one or more of a mathematical function, a condition, a logical function, a machine learning model, natural language processing, and/or image processing. The second plurality of interface components may be sent to the user device 102 via the network interface 128. For example, the one or more processors 123 may send the converted second plurality of interface components to the user device 102.

Application programs and other executable program components such as the operating system 105 are shown herein as discrete blocks, although it is recognized that such programs and components may reside at various times in different storage components of the user device 102, and are executed by the one or more processors 103 of the user device 102. The computing device 122 may include all of the components described with regard to the user device 102.

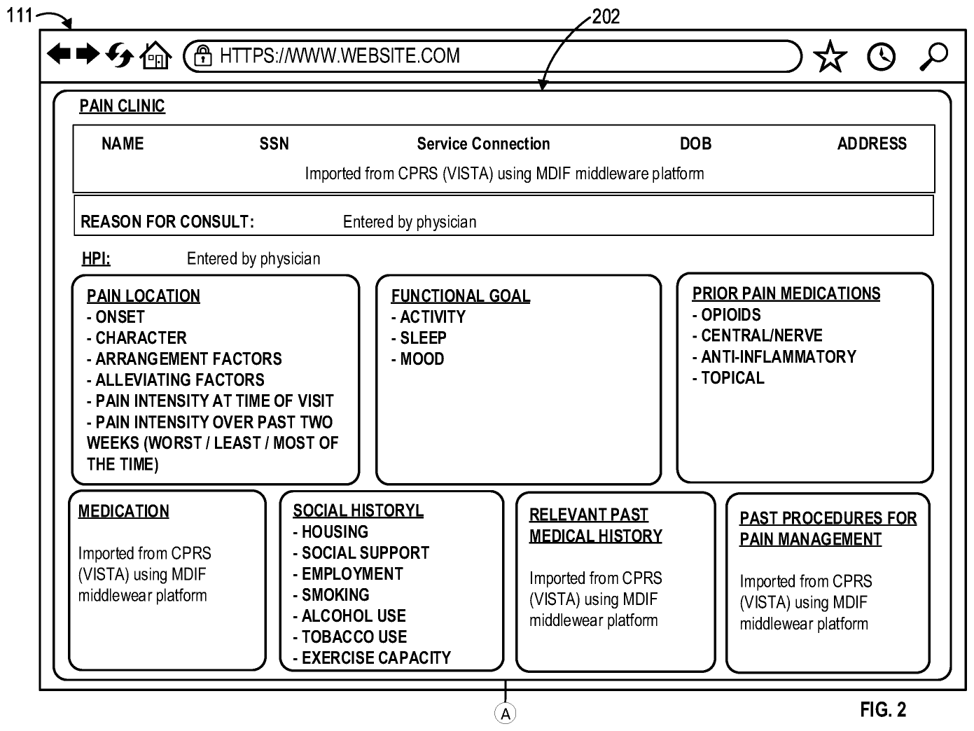

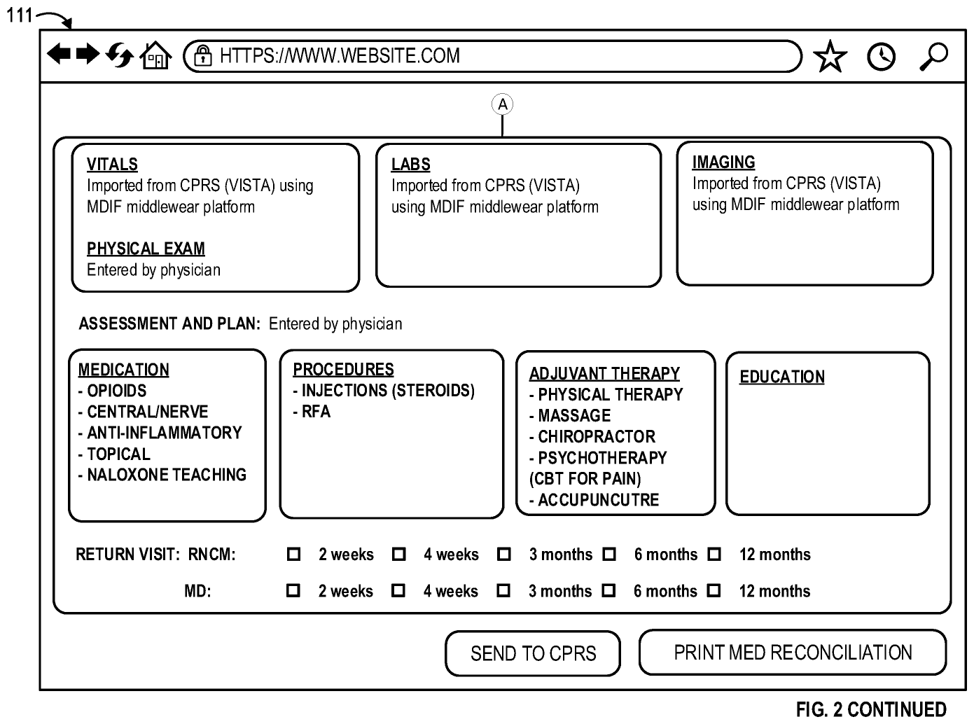

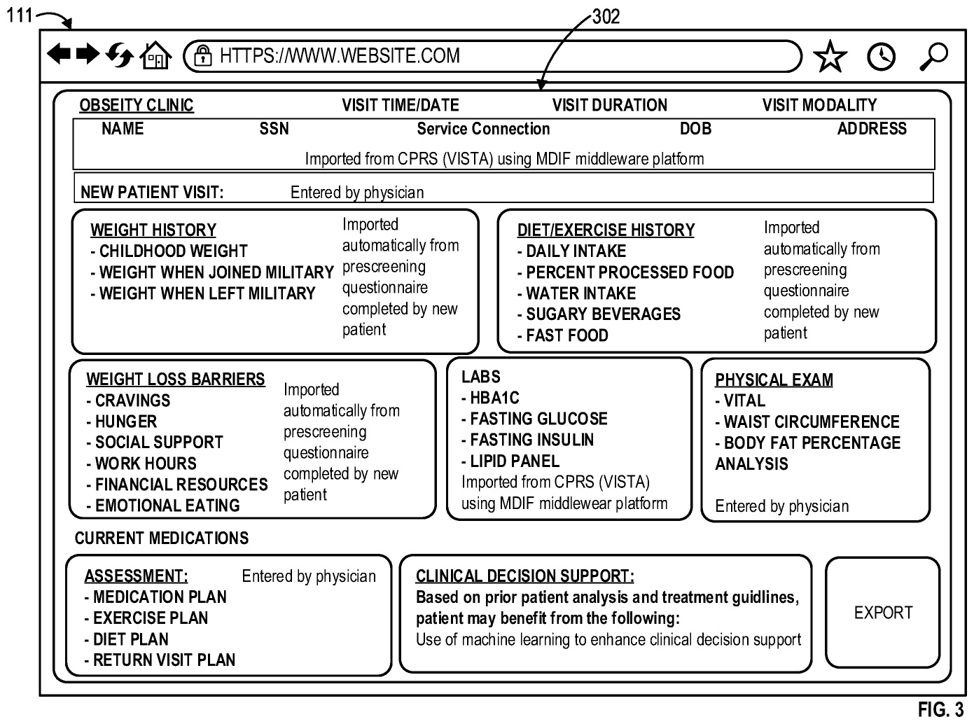

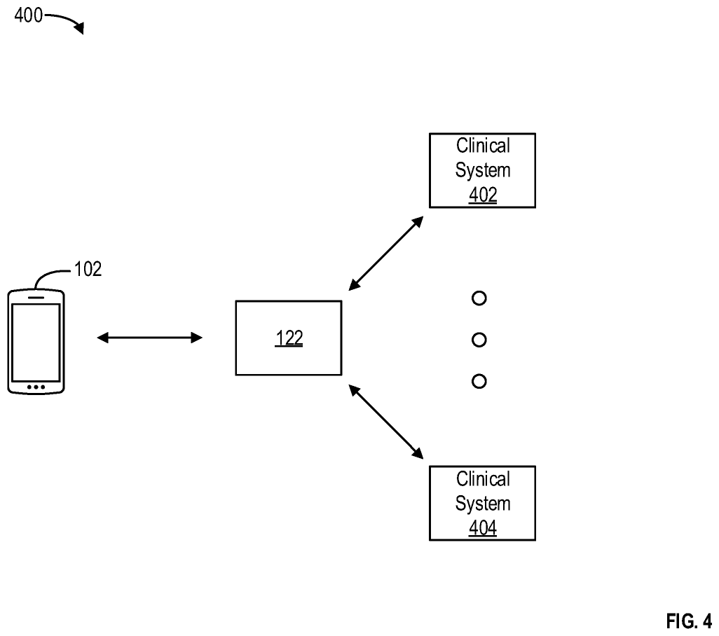

In FIGS. 2-3, the display interface 111 may include interface components 202, 302. Interface components may be content (e.g., text, images, video), borders, framing, infographics, summaries, analytics, indicia, personally identifiable information, or combinations thereof. In some instances, the interface components 202 may include information, or interface components retrieved from one or more clinical systems (e.g., clinical systems 402, 404 shown in FIG. 4). The interface components 202 may include a combination of one or more of information or interface components retrieved from one or more clinical systems. For example, the interface components 202 may be stored on a computing device (e.g., computing device 122) after retrieval from one or more of the clinical systems. The computing device 122 may send one or more interface components 202 and allow one or more of the interface components to be completed by a clinician.

For example, one or more of the interface components 202 may provide a heading and unstructured data related to the heading. For example, one or more interface components 202 may include objects for defining a border, a title, and an area for inputting structured or unstructured data. For example, the structured data may include questions to be answered by the clinician (e.g., radio buttons, true/false questions) and the unstructured data may allow the clinician, or user, to record information in free-form. For example, the unstructured data may be handwritten or typed. An image or video may also be recorded.

The data may be digital. For example, the data may entail text data which may be structured or unstructured. The data may also entail other digital information such as imaging (e.g., radiographs, ECG, ultrasound), sounds (e.g., patient voice notes, clinical staff notes), or other information related to the patient.

For example, the data may based on signals recorded from a patient and processed (e.g., digitally). This information may be displayed based on the underlying patient data as one or more interface components, and patient data may be digital or otherwise. For example, signals may be analyzed from one or more sources (e.g., signal inputs) that vary as a function of time (e.g., an EEG (electroencephalogram) or ECG (electrocardiogram) to extract information (e.g., signal components) that may be of diagnostic value. This analysis may include statistical analysis of the signal or other analytical tools (e.g., time-frequency domain analysis, Fourier transforms, many filtering techniques such as Finite Impulse Response (FIR) filtering, or manipulation of the data obtained from a variety of real-world sources that has been digitized). Such tools may be used individually or in combination.

Image data may be analyzed from one or more sources. This may include artificial intelligence or neural network derived tools used to improve diagnosis. It may also include techniques used for image binarization, segmentation, filtering methods, or combinations thereof. For example, image enhancement, restoration, segmentation, object or feature detection, image to image registration or translation, may be used. Techniques used for images with time domain information may include: motion estimation, motion compensation, and motion vector(s) generation.

While the examples provided herein may be non-exhaustive, these signals and data may be processed (e.g., converted) and used as one or more of the interface components 202, 302. These signals and data may also be combined (e.g., converted) with other data or interfaces to provide a second plurality of interfaces. In such a way, these signals can add diagnostic or therapeutic value to the interface components.

A healthcare organization may segment data in disparate repositories based on practice areas or clinician types (e.g., radiology, internal medicine). Patient data from different parts of the health care organization may be used to form interface components which can then be converted to other interface components using a template or otherwise (e.g., algorithm, intelligent software system, neural net). For example, the platform described herein may allow for the capability to aggregate and covert not only text data but other digital data as well. It does this in a holistic manner for a specific clinic (e.g., obesity, pain). It allows for use of intelligent systems which convert the diverse set of digital data in aggregate to provide actionable information to clinicians.

The display interface 111, for example, may include interface components 202 that pertain to a specific clinic. As shown, the interface components 202 may pertain to the pre-op clinic or a pain clinic. That is, the display interface 111 may be configured to retrieve a list of interface components 202 to populate a specific type of clinic (e.g., pre-operation clinic, pain clinic) from one or more clinical systems. The display interface 111 may be configured to retrieve the list based on a request. As another example, the display interface 111 may further be configured to retrieve a list of interface components to populate another type of clinic (e.g., an obesity clinic). The display interface 111 may display retrieved interface components 202 from the computing device 122. The computing device 122 may retrieve the interface components 202, or interface components to be converted, from one or more clinical systems 402, 404.

The computing device 122 may be configured to convert interface components from the one or more clinical systems to match the look and feel of the display interface 111. For example, clinicians may be unaware that the interface object being interacted with is from a proprietary clinical system. Conversion may be implemented through mathematical functions including but not limited to algebraic, statistical, trigonometric, vectors, and calculus (derivatives). Conditionals (e.g., conditional statements, conditional expressions, conditional constructs) and other commands (e.g., if-then) may be used to convert interface components. A logical function may also be used to convert. The interface components may be converted using machine learning models (e.g., neural networks, decision trees, support vector machines, k-nearest neighbor, random forest, linear regression, logistical regression, Bayesian networks), natural language processing (e.g., text and speech processing, morphological analysis, relational semantics, syntactic analysis, lexical semantics), image processing, or combinations thereof. Further, combinations of processes mentioned herein may be used to convert interface components.

The interface components (e.g., interface components 202, 302) may support clinical operational workflow. For example, different display interfaces 111 may be shown to healthcare workers (HCWs) based on respective roles. That is, display interface 111 may be configured specific to the needs of an attending physician. Display interface 111 may also be configured specific to a resident physician, registered nurse, licensed practical nurses, pharmacist, social worker, psychologist, clinical coordinator, or otherwise. The display interface 111 may include interface components, 202, 302 specific to those used or accessed most often by the specific worker category. Interface components (e.g., interface components 202, 302) may be arranged based on the category or highlighted for a specific user, category of users, or clinical role. Changes to data contained therein may be reflected and updated on the display interface (e.g., display interface 111) seen by other HCWs in real time, enabling a seamless handoff between HCWs during the delivery of clinical care.

For example, the interface components 202, 302 stored at the computing device 122 and sent to the display interface 111 may be converted from interface components of the clinical systems. Conversions may include adjustments to size, shape, coloration, configuration, or combinations thereof. The computing device 122 may include a template or style sheet that defines characteristics of the interface components 202, 302. For example, the template may indicate sizing, shaping, shading, coloration of the interface components 202, 302.

The interface components 202, 302 may include an indicator. For example, one or more indicators may include, but are not limited to, a graph, visualization, infographic, or combination thereof, related to the patient, patient care, or the clinic. The conversion of the interface components 202, 302 may maintain one or more of the indicators from the clinical systems or ensure that the indicators are passed through to the display interface 111.

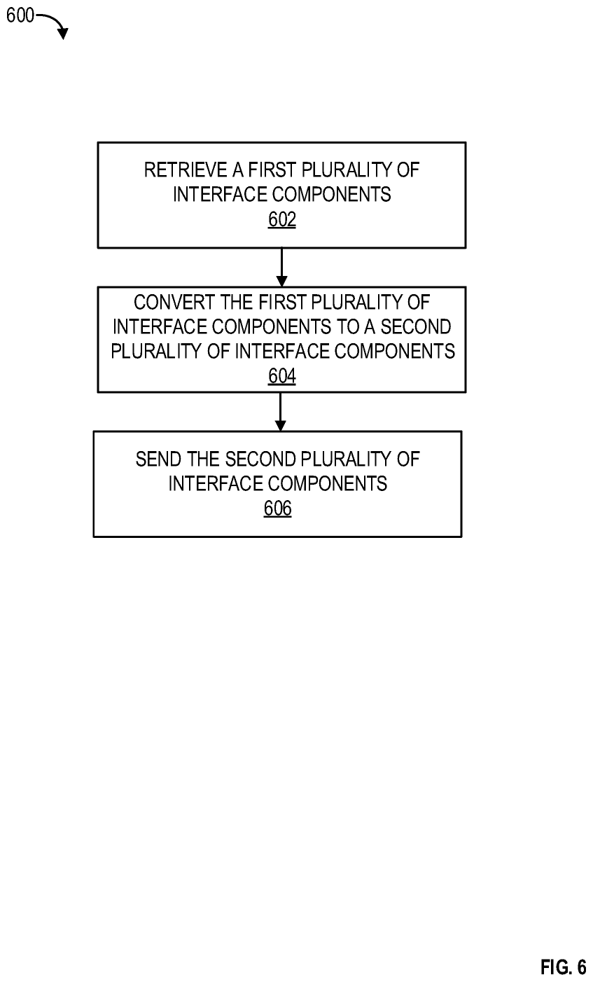

Data displayed on the display interface 111 may be structured (e.g., in rows and columns) or unstructured (e.g., plain text, images). The data may be patient data or practitioner data. The data may be entered by the patient, entered by the clinician, or a combination thereof. A neural network, e.g., a neural network trained in FIG. 6, may be used to determine one or more interface components 202, 302 or other information. The neural network may be deep. The neural network may include parameters (e.g., weights), nodes, and edges, to make determinations based on the data. For example, the neural network may perform natural language processing on the structured or unstructured data. For example, the neural network may be trained to make determinations based on unstructured text data from one or more of the clinical systems. The neural network may be trained to perform natural language processing on text data and perform question answering.

The neural network may be retrieved from the computing device 122. For example, the neural network may be retrieved from the computing device based on a request for the interface components 202, 302. The interface components 202, 302 may be determined based on the neural network.

In FIG. 4 an example system 400 in accordance with one or more implementations of the present disclosure is shown. For example, the system may include clinical systems 402, 404. The clinical systems 402, 404 may be networked or in communication with the computing device 122, the user device 102, or a combination thereof. The clinical systems 402, 404 may include similar components (e.g., processors, instructions, computer-readable mediums) to those of the user device 102 and/or the computing device 122.

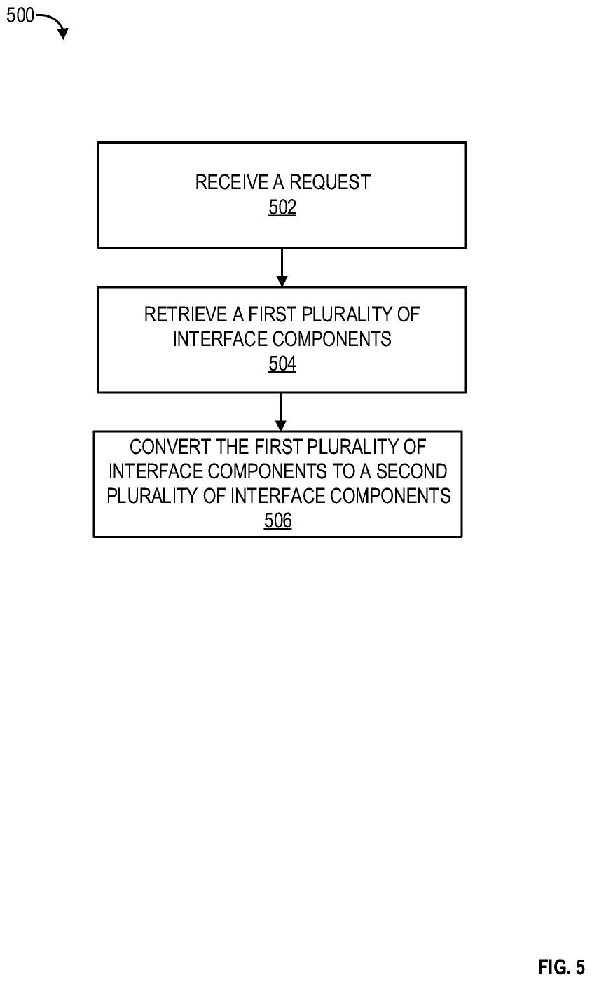

FIG. 5 an example method in accordance with one or more implementations of the present disclosure is shown. The method 500 may be performed by the user device 102, the computing device 122, the clinical systems 402, 404, or a combination thereof. In step 502, a request may be received related to one or more of the clinical systems 402, 404. For example, the request may be a request for one or more interface components. The request may be sent to the computing device 122. For example, the user device 102 and/or the clinical systems 402, 404 may send the request to the computing device 122.

In step 504, one or more of the interface components may be retrieved. The request may cause the computing device 122 to retrieve a first plurality of interface components from the clinical systems 402, 404. The computing device 122 may retrieve the first plurality of interface components from computer storage associated with the clinical systems 402, 404 in real-time. The computer storage may comprise a plurality of interface components that correspond to a plurality of clinical systems including the clinical systems 402, 404. The first plurality of interface components may comprise content such as text, images, and video. The first plurality of interface components may comprise borders, framing, infographics, summaries, analytics, indicia, personally identifiable information, and/or the like. The first plurality of interface components may comprise patient data. The patient data may include structured data and/or unstructured data. The structured data may include questions to be answered by clinicians, users, or physicians. For example, radio buttons and/or true/false questions may be the form of structured data. The unstructured data may allow the clinicians or physicians to enter patient information in free-form. The unstructured data may comprise one or more of a radiograph, ECG, ultrasound, and/or textual data. The unstructured data may comprise one or more of patient voice notes, clinical staff notes, and textual data.

In step 506, the computing device 122 may convert the first plurality of interface components to a second plurality of interface components (e.g., interface components 202, 302). The first plurality of interface components may be converted to the second plurality of interface components based on a template associated with the computing device 102. The template may indicate sizing, shaping, shading, and coloration of the second plurality of interface components. Thus, the conversion from the first plurality of interface components to the second plurality of interface components may include one or more of adjustments to size, shape, coloration, and configuration. The template may further comprise one or more of a mathematical function, a condition, a logical function, a machine learning model, natural language processing, and image processing.

The first plurality of interface components may comprise one or more indicators. Examples of the indicators may include, but are not limited to, a graph, visualization, and infographics related to the patient or the clinic. The one or more indicators may be preserved in the conversion of the first plurality of interface components to the second plurality of interface components such that the one or more indicators are present in the second plurality of interface components. The second plurality of interface components may then be sent. For example, the computing device may send the converted second plurality of interface components to the user device 102 or the clinical systems 402, 404.

FIG. 6 an example method in accordance with one or more implementations of the present disclosure is shown. The method 600 may be performed by the user device 102, the computing device 122, the clinical systems 402, 404, or a combination thereof. In step 602, a first plurality of interface components may be retrieved. For example, the computing device 122 may retrieve the first plurality of interface components from its data storage. The first plurality of interface components may be received from the clinical systems 402, 404 and saved in the data storage of the first computing device 122. The first plurality of interface components may be received from each of the plurality of clinical systems (including the clinical systems 402, 404) and retrieved from the data storage in real-time. A request from the user device 102 may cause the computing device 122 to retrieve the first plurality of interface components. The first plurality of interface components may comprise patient data associated with the plurality of clinical systems. Examples of the plurality of clinical systems may include, but are not limited to, an obesity clinic system, a heart failure clinic system, a chronic kidney disease clinic system, and a pain clinic system.

The first plurality of interface components may comprise content such as text, images, and video. The first plurality of interface components may comprise borders, framing, infographics, summaries, analytics, indicia, personally identifiable information, and/or the like. The first plurality of interface components may comprise patient data. The patient data may include structured data and/or unstructured data. The structured data may include questions to be answered by clinicians, users, or physicians. For example, radio buttons and/or true/false questions may be the form of structured data. The unstructured data may allow the clinicians or physicians to enter patient information in free-form. The unstructured data may comprise one or more of a radiograph, ECG, ultrasound, and/or textual data. The unstructured data may comprise one or more of patient voice notes, clinical staff notes, and textual data.

In step 604, the first plurality of interface components may be converted to a second plurality of interface components. For example, the computing device 122 may convert the first plurality of interface components to the second plurality of interface components. The second plurality of interface components may be associated with a clinical system of the plurality of clinical systems. The first plurality of interface components may be converted to the second plurality of interface components based on a template associated with the clinical system. The first plurality of interface components may be converted to the second plurality of interface components based on a template, a style sheet, an algorithm, an intelligent software system, machine learning, a neural network, or the like associated with the clinical system. For example, the computing device 122 may convert the first plurality of interface components to the second plurality of interface components based on a machine learning model. The machine learning model may be configured to determine the second plurality of interface components based on the structured data and/or the unstructured data.

The first plurality of interface components may comprise one or more indicators. Examples of the indicators may include, but are not limited to, a graph, visualization, and infographics related to the patient or the clinic. The one or more indicators may be preserved during the conversion of the first plurality of interface components to the second plurality of interface components. For example, the one or more indicators may be maintained or present in the second plurality of interface components after the conversion.

In step 606, the second plurality of interface components may be sent. For example, the computing device 122 may send the second plurality of interface components to the user device 102 or the clinical systems 402, 404. The second plurality of interface components may be used to display the patient data that is compatible with the clinical system. For example, the first plurality of interface components retrieved from multiple clinical systems (e.g., an obesity clinic system, a heart failure clinic system, a chronic kidney disease clinic system) may be converted to the second plurality of interface components that are used to display the patient data for a specific clinical system (e.g., an obesity clinic system). The second plurality of interface components may be used to generate the display of the patient data in the clinical system of the plurality of the clinical system by the user device 102 or the clinical systems 402, 404.

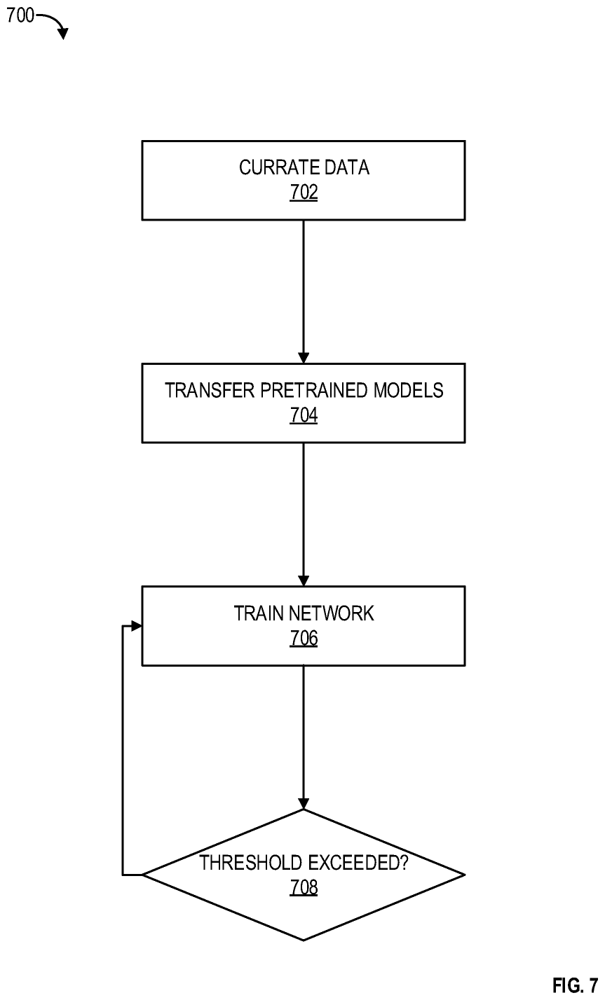

In FIG. 7, an example method 700 for training one or more networks (e.g., a neural network, another machine learning algorithm) in accordance with one or more implementations of the present disclosure is shown. The method may be performed on one or more computing systems described herein (e.g., computing device 122, another computing device, cloud computing).

The method 700 includes curation of training data and testing data in step 702. For example, data for training the neural network may include examples of structured or unstructured data. The training may be supervised with correct answers and a question answering service. The neural network may be a transformer or otherwise, or combinations thereof. The training data may be divided to reserve test data for measuring the accuracy of the determinations by the neural network.

In step 704, the neural network may be pre-trained. For example, the neural network may be pre-trained on generic unstructured text data and transferred for additional specific learning for the task. In step 706, the neural network, or individual neural networks (e.g., ensemble networks) may be trained according to the training data described herein until an error threshold is exceeded.

In step 708, the neural network may be evaluated. The error may be different depending on the data trained and acceptable thresholds may be different depending on the data trained. Once the error threshold is exceeded, the neural network may be considered trained.

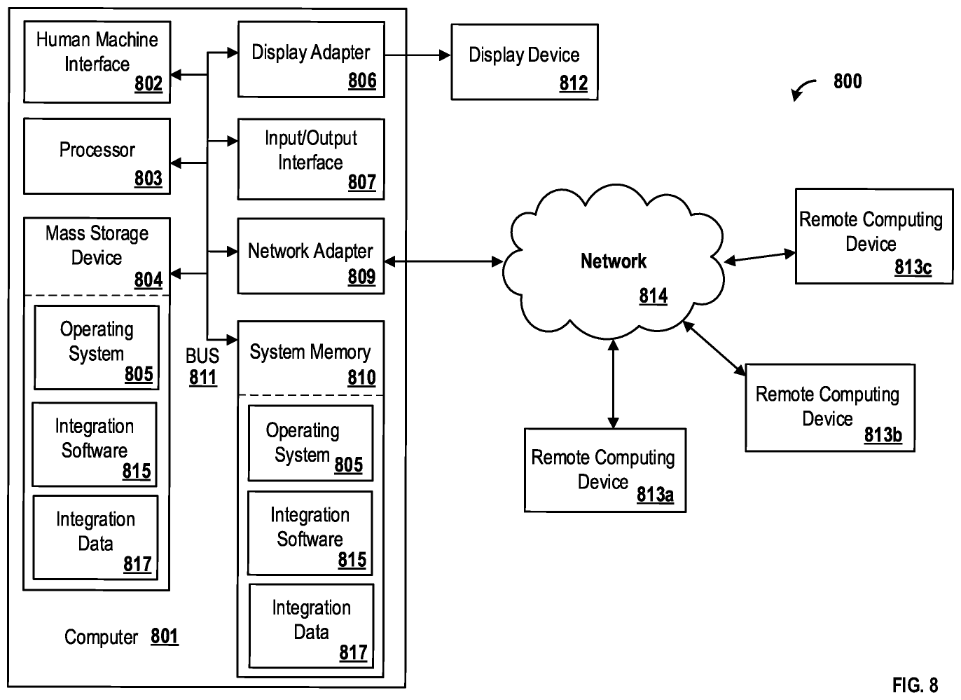

In an exemplary aspect, the methods, apparatuses, and systems can be implemented on a computer 801 as illustrated in FIG. 8 and described below. By way of example, the user device 102, and the computing device 122 of FIG. 1 can be a computer 801 as illustrated in FIG. 8. The clinical systems 402, 404 can be a computer 801 as illustrated in FIG. 8. Similarly, the methods, apparatuses, and systems disclosed can utilize one or more computers to perform one or more functions in one or more locations. For example, the computer 801 may perform or implement the methods or processes described in FIGS. 2-6. By way of example, remote computing devices 813a-c can be clinical systems 402, 404. A healthcare network may comprise the computer 801 and the remote nodes 813a-c. The computer 801 and the remote nodes 813a-c may form a set of network topologies in the healthcare network. FIG. 8 is a block diagram illustrating an exemplary operating environment 800 for performing the disclosed methods. This exemplary operating environment 800 is only an example of an operating environment and is not intended to suggest any limitation as to the scope of use or functionality of operating environment architecture. Neither should the operating environment 800 be interpreted as having any dependency or requirement relating to any one or combination of components illustrated in the exemplary operating environment 800.

The present methods, apparatuses, and systems can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, and multiprocessor systems. Additional examples comprise programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, and/or the like that perform particular tasks or implement particular abstract data types. The disclosed methods can also be practiced in grid-based and distributed computing environments where tasks are performed by remote processing devices that are linked through a communications network. In a distributed computing environment, program modules can be located in local and/or remote computer storage media such as memory storage devices.

Further, one skilled in the art will appreciate that the systems, apparatuses, and methods disclosed herein can be implemented via a general-purpose computing device in the form of a computer 801. The computer 801 can comprise one or more components, such as one or more processors 803, a system memory 810, and a bus 811 that couples various components of the computer 801 comprising the one or more processors 803 to the system memory 810. The system can utilize parallel computing.

The bus 811 can comprise one or more of several possible types of bus structures, such as a memory bus, memory controller, a peripheral bus, an accelerated graphics port, or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 811, and all buses specified in this description can also be implemented over a wired or wireless network connection and one or more of the components of the computer 801, such as the one or more processors 803, a mass storage device 804, an operating system 805, a network adapter 808, the system memory 810, an Input/Output Interface 807, a display adapter 806, a display device 812, and a human machine interface 802, can be contained within one or more remote computing devices 813a,b,c at physically separate locations, connected through buses of this form, in effect implementing a fully distributed system.

The computer 801 comprises a variety of computer readable media. Exemplary readable media can be any available media that is accessible by the computer 801 and comprises, for example and not meant to be limiting, both volatile and non-volatile media, removable and non-removable media. The system memory 810 can comprise computer readable media in the form of volatile memory, such as random access memory (RAM), and/or non-volatile memory, such as read only memory (ROM). The system memory 810 can comprise data such as the operating system 805, clinical integration software 815, and clinical integration data 817 that are accessible to and/or is operated on by the one or more processors 803. The clinical integration software 815 may include data to perform or implement the methods or processes described in FIGS. 2-6. The clinical integration data 817 may be a database to perform or implement the clinical integration software 815. For example, the clinical integration data 817 may comprise a plurality of interface components associated with the remote computing devices 813a-c.

In an embodiment, the computer 801 can also comprise other removable/non-removable, volatile/non-volatile computer storage media. The mass storage device 804 can provide non-volatile storage of computer code, computer readable instructions, data structures, program modules, and other data for the computer 801. For example, the mass storage device 804 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Additionally, any number of program modules can be stored on the mass storage device 804, such as, by way of example, the operating system 805, clinical integration software 815, and clinical integration data 817. The operating system 805 can comprise elements of the programming and be stored on the mass storage device 804. Examples of such databases comprise, DB2®, Microsoft® Access, Microsoft® SQL Server, Oracle®, mySQL, PostgreSQL, and the like. The databases can be centralized or distributed across multiple locations within the network 814. The clinical integration software 815 may include data to perform or implement the methods or processes described in FIGS. 2-6. The clinical integration data 817 may be database to perform or implement the clinical integration software 815. For example, the clinical integration data 817 may comprise a plurality of interface components associated with a plurality of clinical systems (e.g., the remote computing devices 813a-c).

In an embodiment, the user can enter commands and information into the computer 801 via an input device (not shown). Examples of such input devices comprise, but are not limited to, a keyboard, a pointing device (e.g., a computer mouse, remote control), a microphone, a joystick, a scanner, tactile input devices such as gloves, and other body coverings, motion sensor, and the like These and other input devices can be connected to the one or more processors 803 via the human machine interface 802 that is coupled to the bus 811, but can be connected by other interface and bus structures, such as a parallel port, game port, an IEEE 1394 Port (also known as a Firewire port), a serial port, a network adapter 809, and/or a universal serial bus (USB).

In an embodiment, the display device 812 can also be connected to the bus 811 via an interface, such as the display adapter 806. It is contemplated that the computer 801 can have more than one display adapter 806 and the computer 801 can have more than one display device 812. For example, the display device 812 can be a monitor, an LCD (Liquid Crystal Display), light emitting diode (LED) display, television, smart lens, smart glass, and/or a projector. In addition to the display device 812, other output peripheral devices can comprise components such as speakers (not shown) and a printer (not shown) which can be connected to the computer 801 via an Input/Output Interface 807. Any step and/or result of the methods can be output in any form to an output device. Such output can be any form of visual representation, comprising, but not limited to, textual, graphical, animation, audio, tactile, and the like. The display device 812 and the computer 801 can be part of one device, or separate devices.

The computer 801 can operate in a networked environment using logical connections to one or more remote computing devices 813a,b,c. By way of example, a remote computing device 813a,b,c can be a personal computer, computing station (e.g., workstation), clinical computing system, portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network nodes, and so on. The remote computing device 813a,b,c may be a node (e.g., clinic or hospital) in a healthcare network. Logical connections between the computer 801 and a remote computing device 813a,b,c can be made via a network 814, such as a local area network (LAN) and/or a general wide area network (WAN). Such network connections can be through the network adapter 809. The network adapter 809 can be implemented in both wired and wireless environments. Such networking environments are conventional and commonplace in dwellings, offices, enterprise-wide computer networks, intranets, and the Internet.

For purposes of illustration, application programs and other executable program components such as the operating system 805 are illustrated herein as discrete blocks, although it is recognized that such programs and components can reside at various times in different storage components of the computing device 801, and are executed by the one or more processors 803 of the computer 801. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

For purposes of illustration, application programs and other executable program components are illustrated herein as discrete blocks, although it is recognized that such programs and components can reside at various times in different storage components. An implementation of the described methods can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

While the methods and systems have been described in connection with specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A system comprising:
a computer store containing data, for each of a plurality of clinical systems, defining a first plurality of interface components that correspond to the plurality of clinical systems; and
a computer server configured to:
receive a request based on one or more of the plurality of clinical systems;
retrieve the first plurality of interface components; and
convert the first plurality of interface components to a second plurality of interface components corresponding to the computer server.

2. The system of claim 1, wherein the first plurality of interface components are converted to the second plurality of interface components based on a template of the computer server.

3. The system of claim 2, wherein the first plurality of interface components comprise an indicator, wherein the indicator is preserved in the conversion of the first plurality of interface components to the second plurality of interface components such that the indicator is present in the second plurality of interface components.

4. The system of claim 1, wherein the first plurality of interface components are retrieved from each of the plurality of clinical systems in real-time.

5. The system of claim 1, wherein the first plurality of interface components comprise patient data, wherein the patient data comprise structured data and unstructured data.

6. The system of claim 5, wherein the unstructured data is based on an entry associated with the second plurality of interface components.

7. The system of claim 5, wherein the patient data comprise first data associated with a first clinical system of the plurality of clinical systems and second data associated with a second clinical system of the plurality of clinical systems.

8. The system of claim 7, wherein the patient data is based on a questionnaire completed by a patient.

9. The system of claim 1, wherein the computer server is further configured to:
receive patient data based on one of the second plurality of interface components, wherein the patient data is further based on one of the plurality of clinical systems; and
cause the patient data to be stored in the one of the plurality of clinical systems.

10. A method comprising:
receiving a request based on one or more of a plurality of clinical systems;
retrieving a first plurality of interface components; and
converting the first plurality of interface components to a second plurality of interface components.

11. The method of claim 10, wherein the first plurality of interface components are converted to the second plurality of interface components based on a template.

12. The method of claim 11, wherein the template comprises one or more of a mathematical function, a condition, a logical function, a machine learning model, natural language processing, or image processing.

13. The method of claim 11, wherein the first plurality of interface components comprise an indicator, wherein the indicator is preserved in the conversion of the first plurality of interface components to the second plurality of interface components such that the indicator is present in the second plurality of interface components.

14. The method of claim 11, wherein the first plurality of interface components are retrieved from each of the plurality of clinical systems in real-time.

15. A method comprising:
retrieving, based on a plurality of clinical systems, a first plurality of interface components, wherein the first plurality of interface components comprise patient data associated with the plurality of clinical systems;
converting the first plurality of interface components to a second plurality of interface components associated with a clinical system of the plurality of clinical systems; and
sending the second plurality of interface components for displaying the patient data in the clinical system.

16. The method of claim 15, wherein the patient data comprise structured data and unstructured data.

17. The method of claim 16, wherein the unstructured data is based on an entry associated with the second plurality of interface components.

18. The method of claim 16, wherein the first plurality of interface components are converted to the second plurality of interface components using a machine learning model configured to determine the second plurality of interface components based on the unstructured data.

19. The method of claim 15, further comprising:
receiving, from a user device, a request for the second plurality of interface components.

20. The method of claim 15, wherein the first plurality of interface components are retrieved from each of the plurality of clinical systems in real-time.
